(19) United States
(12) Patent Application Publication
(10) Pub. No.: US 2016/0106958 A1
(43) Pub. Date: Apr. 21, 2016

(54) An Intravenous Cannula Retainer

(71) Applicant: MARTELLUS PTY LTD, Hornsby Heights, NSW (AU)

(72) Inventor: Roger PRICE, Hornsby Heights (AU)

(73) Assignee: ResQDevices Pty Ltd, Eveleigh (AU)

(21) Appl. No.: 14/889,305

(22) PCT Filed: May 5, 2014
(86) PCT No.: PCT/IB2014/000682

§ 371 Date: Nov. 5, 2015

(30) Foreign Application Priority Data
May 5, 2013 (AU) 2013901588

(51) Int. Cl. A61M 25/02

(52) U.S. Cl. CPC A61M 25/02; A61M 2025/0206; A61M 2025/0233; A61M 2025/024; A61M 2025/0253

(57) ABSTRACT

An intravenous cannula retainer is described. The intravenous cannula comprises a cuff suitable for at least partially encircling at least a portion of a limb of a patent, the cuff including at least one intravenous cannula retaining zone. A method of cannulation is also described.

### CROSS-REFERENCE TO RELATED APPLICATIONS

This application is a US 371 from PCT/IB2014/000682 filed May 5, 2014, which claims priority to Australia Application No. 2013901588 filed May 5, 2013, the technical disclosures of which are herein incorporated by reference.

### FIELD OF THE INVENTION

The invention relates generally to an intraarterial or intravenous cannula retainer. Embodiments of the intravenous cannula retainer may find particularly effective application in paediatric care where intravenous cannulation is required. Other effective applications are on animals in veterinary care, and in adults who may be unwilling or unable to cooperate with treatment, such as ford example aged patients.

### BACKGROUND OF THE INVENTION

Intraarterial and intravenous cannulation can improve outcomes during prehospital and hospital treatment of patients by facilitating the intraarterial and intravenous administration of fluids and drugs to patients.

Intravenous cannulation can be traumatic, both during introduction of the intravenous cannula and while it is in place. Some patients are very sensitive to the introduction process and some are very sensitive even to the mere presence of an intravenous cannula in their body. These sensitive patients are mainly children but many are adults.

The trauma caused by intravenous cannulation in sensitive patients can cause that patient to try to remove the intravenous cannula from their person. This can delay treatment and positive outcomes.

The present invention seeks to ameliorate one or more of the abovementioned disadvantages and/or seeks to provide a carer with a new cannula retainer.

In this specification, where a document, act or item of knowledge is referred to or discussed, this reference or discussion is not an admission that the document, act or item of knowledge or any combination thereof was at the priority date:
part of common general knowledge; or
known to be relevant to an attempt to solve any problem with which this specification is concerned.

### Definitions

Throughout this specification and the claims that follow, including the drawings, the following definitions shall apply:

Cannula: A thin tube, including tube attachments, inserted into a vein or body cavity to administer medicine, drain off fluid, or insert a surgical instrument. Except where otherwise noted above, the terms intravenous and intraarterial are generally considered to be coterminous throughout the specification and claims.

### SUMMARY OF THE INVENTION

In accordance with a broad form of the invention there is provided an apparatus for inhibiting access to an intravenous cannula site, or an intravenous cannula disposed within a blood vessel of a patient and thereby facilitating the retention of the cannula in the limb and inhibiting removal of the cannula from the blood vessel of the patient.

Advantageously, preferred embodiments of the present invention mitigate the impact of pulling on an IV line and inhibiting its removal.

In accordance with one aspect of the present invention there is provided an intravenous cannula retainer comprising a cuff suitable for at least partially supporting at least a portion of a limb of a patient, the cuff including at least one intravenous cannula retaining zone.

Optionally the cuff is in the form of a splint for immobilising at least a portion of the limb.

In one optional form the splint includes a support suitable for supporting the limb of the patient in one or more selected limb positions. For example, a finger and/or wrist and/or elbow could be supported in a straight position, an extended position or a flexed position. Optionally the support includes a support curtain for at least partially enveloping the limb of the patient in each of the one or more selected positions. The support may include battens or other elongate members to facilitate support of the limb in the one or more selected straight, extended or flexed limb positions.

Preferably the support facilitates the immobilisation of the limb in each of the one or more selected extended or flexed or straight positions. Preferably one of the selected positions includes a position wherein a cannula can be inserted into a vein extending along the limb. Preferably another of the selected positions includes a limb position in which fluid freely flows through the cannula and the limb is comfortable.

Preferably the support curtain includes reinforcing elements. The reinforcing elements may include battens or other elongate elements. Preferably the reinforcing elements include granules. Preferably the support curtain includes at least two layers of curtain material and a splint void therebetween. Preferably the curtain material is a tear-resistant polymer. Preferably the curtain material is strengthened by strengthening elements such as fibres. Preferably the granules are disposed in the void between at least two of the at least two layers of curtain material.

Preferably the curtain material is airtight so as to be able to hold a vacuum in the splint void between at least two of the at least two layers of curtain material. Preferably the support curtain includes a valve in order to facilitate extraction of air from the splint void between at least two of the at least two layers of support curtain material in order to cause the granules to abut one another and to reduce movement between the granules once they are in an abutting relationship. This arrangement in use facilitates immobilisation of the limb in the one or more selected positions by inhibiting flexibility of the support curtain.

Preferably the support curtain includes a closure to close the support curtain about the limb in the selected position. Preferably the closure includes one or more fasteners to fasten one side of the support curtain to another side of the support curtain. Preferably the support curtain includes a thumb spice to support and/or immobilise the thumb in a support position.

Preferably the fasteners include a locking to inhibit their release by the patient whose limb is immobilised and wearing the cannula retainer.

Preferably the lock includes elongate straps extending from the one side of the support curtain, Optionally the elongate straps are rigid. Optionally the elongate straps include hook and/or loop fasteners on one side thereof for fastening to hook and/or loop fasteners on the other side thereof. Preferably the elongate straps are integral with the support curtain material, the arrangement being that the straps include two layers of the support curtain material and are filled with granules in a strap void therebetween, the strap void being in fluid communication with the splint void. The advantage of the strap having a strap void and hook and loop fasteners on one side is that the strap is increased in rigidity and in use is inhibited from being peeled away from the support curtain, which forms a kind of locking fastener. When air is reintroduced back into the strap void, the strap becomes flexible again and is less of a hazard to the patient and the carer.

Preferably there is provided a liner to facilitate patient comfort when the limb is immobilised in the splint. Preferably the liner includes a liner penetration for the thumb. Preferably the liner includes retention zones or includes a retention cover such that the liner can be retained in a selected lining position in the support curtain. Preferably the liner includes a lining substrate of memory foam, or foam, or some resilient support means to inhibit compartment syndrome when the splint is in a support or immobilisation position.

Optionally, the intravenous cannula retaining zone includes a cannula retaining means. Preferably the cannula retaining means includes a cannula mount. Optionally the cannula mount includes a cannula penetration for allowing a cannula to penetrate the support curtain. Preferably the cannula penetration is an aperture. Preferably the aperture is about 100 mm×100 mm in size and extends from one face of the support curtain to the other.

Preferably the cannula mount includes a cannula penetration closure. Preferably the cannula penetration closure includes a closure rim for at least partially surrounding the rim of the aperture.

Preferably the penetration closure includes a cover for covering the cannula penetration. Preferably the cover includes a window cap. Preferably the window cap is transparent or translucent to allow inspection of the patient's limb disposed in use under the aperture. Preferably the cap is a cup-shape to provide some volume under the window cap.

Preferably the window cap includes a window cap retainer. Preferably the window cap retainer includes a keeper. Preferably the keeper is adapted to be received in a catch disposed on the closure rim. Preferably the keeper is removably connectable to the catch and the arrangement is such that the keeper can be operated by a child-resistant lock. In one preferred form the child-resistant lock includes a flexible keeper arm and a rib extending therefrom so as to be caught by the catch in a locking position. The catch includes a cross-member so that the rib can be released from the catch only by a finger press under the cross-member, the cross-member being hidden from view from a child.

Preferably the window cap retainer includes locating lugs to facilitate locating the window cap on the rim.

In one embodiment the cannula penetration closure includes a cannula mounting base.

Optionally the rim or the cannula mounting base includes a cannula rest for resting a portion of the cannula thereon.

Optionally the cannula rest is disposed flat and against the skin of the patient so that the cannula may remain flat and horizontal so as to inhibit breaking through a wall of a blood vessel when the cannula is on the cannula rest. Optionally the cannula rest extends from a portion of the rim and in one embodiment is integral therewith, so that in use a wall of the window cap when in the closed position can inhibit release of the cannula from the cannula rest. Optionally the cannula rest includes a cannula rest cradle to facilitate immobilisation and support of the cannula when in use.

Optionally the rim includes locating lug receivers to receive the locating lugs and facilitate locking and retention of the window cap.

Optionally the penetration closure includes a cannula rest cover for covering the cannula rest. Optionally the cannula rest cover includes a cannula rest cap and is preferably transparent or translucent to facilitate monitoring of the retention of the cannula.

Optionally the cannula mounting base includes a cannula tube receiver. Preferably the cannula tube receiver includes a tube mount. Preferably the tube mount extends along one portion of the rim. Preferably the tube mount includes a tube channel for mounting tube therealong. Preferably the tube channel includes a break so as to facilitate locking of the tube within the tube mount. Preferably the tube channel includes one or more retaining ribs along an open edge so as to retain the tube within the channel. Optionally the tube channel is a sinusoidal or sawtooth channel or in the form of discrete tube supports disposed in a sinusoidal arrangement on the support curtain so that the tube is directed to a sinusoidal position.

Preferably a line lock is provided to lock the tube within the tube channel. Preferably the line lock includes a main lock body including a tube receiver and a locking means. Preferably the locking means includes at least one resilient locking member. Preferably the tube receiver includes a channel. Preferably the resilient locking member includes an actuator and a detent. Preferably the resilient locking member includes a sigmoid element so that the base of the sigmoid can be received by the tube channel or a detent receiver and the top of the sigmoid can be the actuator to retract the detent or the base of the sigmoid from the tube channel.

In one embodiment the support curtain extends along a limb far enough to provide immobilisation of a wrist. In that embodiment, the cannula penetration is disposed on a rear portion of the support curtain so that the cannula penetration is disposed adjacent the back of a patient's hand when in an immobilising position on the patient's limb.

In another embodiment the support curtain extends along the limb far enough to provide immobilisation of an elbow. In yet another embodiment the support curtain extends far enough along the limb to provide immobilisation of an elbow, arm and wrist joints.

In one embodiment a cannulation assembly is provided. Preferably the cannulation assembly includes a tourniquet assembly. Preferably the tourniquet assembly includes a belt for encircling the arm of the patient and restricting blood flow through the arm past the tourniquet. Preferably a tourniquet actuation means is provided. Preferably the tourniquet actuation means includes a belt advancing means to tighten the belt around the arm. Preferably the belt advancing means includes a dial which twists to advance the belt through the belt advancing means.

Preferably the tourniquet assembly includes a tourniquet release. Preferably the tourniquet release is disposed on the dial. Preferably the tourniquet release is actuated by pressing the dial, in a similar way to certain kinds of ball-point pen retraction means.

The tourniquet release may be in an interlock form, in which a tourniquet release arm is actuated by a tourniquet release arm disposed on the penetration closure.

Preferably the cannula penetration cover includes a tube channel cover. Preferably the tube channel cover includes an elongate cover wall extending along the tube channel from a rear portion of the cannula rest cover to an end of the rim.

According to yet another aspect of the present invention there is provided a method of retaining a cannula in a patient.

In accordance with a yet further aspect of the present invention there is provided a fastener suitable for use with a cuff or splint, the fastener including a member which is adapted to adopt a rigid disposition and a flexible disposition.

Preferably the member includes an airtight void within which granules or other short elongate inserts are disposed. Preferably the member includes outer layers of airtight material surrounding the void. Preferably the member is elongate. Preferably the elongate member is a strap.

Preferably a valve is disposed on an outer layer for facilitating fluid communication with ambient atmosphere outside the airtight void. Preferably the elongate member includes a hook and loop fastener disposed along one side of the member.

To operate, the hook and loop elements of the strap are connected to their corresponding hook and loop elements on an abutting surface. At this point, they are fastened but may be easily parted by simply peeling the strap away. Then air is withdrawn from the elongate strap so that the granules or other elongate inserts in the void abut one another and are inhibited from movement relative to one another. This action causes the strap to adopt a rigid disposition which inhibits peeling of the strap. That rigid disposition causes the hook and loop fasteners disposed on the strap to be very strongly held to one another and very difficult to part.

In accordance with still another aspect of the present invention there is provided a method of retaining a cannula in a limb of a patient, the method including the steps of immobilising the limb with a splint and mounting at least a portion of the cannula on the splint to inhibit access and/or release by a patient.

In accordance with a still yet further aspect of the present invention there is provided a method of cannula introduction, the method including the steps of:
a. applying a cuff to a portion of a limb of a patient adjacent a cannulation site;
b. applying a tourniquet to the limb;
c. introducing the cannula to a blood vessel of the limb;
d. inhibiting access to the cannulation site with a closure; and
e. releasing the tourniquet with the closure.

In accordance with another further aspect of the present invention there is provided a cannulation apparatus which includes a tourniquet release. Preferably the cannulation apparatus includes a cuff. Preferably the cuff includes an integral tourniquet. Preferably the cuff includes an aperture to provide access to a cannulation site. Preferably the cuff includes a closure to close the aperture. Preferably the closure includes a tourniquet release which upon application of the closure to the aperture, releases the tourniquet.

It is to be understood that although there are several distinct aspects of the invention defined individually herein, they may be combined with one another so that optional aspects of the invention may be provided in combination with any one aspect hereindefined and described.

### BRIEF DESCRIPTION OF THE DRAWINGS

To assist with understanding the invention, it will now be explained with reference to preferred embodiments illustrated in the accompanying drawings in which:

FIG. 1 is an isometric view of a wrist cannula retainer in accordance with a preferred embodiment of the present invention, the wrist cannula retainer shown in a retention position on a wrist of a patient;

FIG. 2 is an isometric view of the wrist cannula of FIG. 1 in a standby position, unwrapped and ready to apply to a wrist of a patient;

FIG. 3 is a detail view of a portion of the embodiment shown in FIG. 1, showing an isometric view of a line lock;

FIG. 4 is a plan view of a liner for use in the embodiment of FIG. 1;

FIG. 5 is a plan view of an arm cannula retainer in accordance with another preferred embodiment of the present invention, the arm cannula retainer shown in a standby position, ready to be placed on an arm of a patient;

FIG. 6 is an isometric view of the arm cannula retainer shown in FIG. 5, the arm cannula retainer shown in a retaining position;

FIG. 7 is a detail view of a portion of FIG. 6, the portion being a tube channel adjacent a window cup, the tube channel for retaining a tube of the intravenous cannula;

FIG. 8 is a plan view of an arm cannula retainer liner disposed in a standby position ready for application to the underside of the arm cannula retainer shown in FIG. 5;

FIG. 9 is an isometric view of a cannulation assembly in the form of a tourniquet assembly, the tourniquet assembly being a component of a preferred embodiment of the present invention;

FIG. 10 is a side elevation section view of the tourniquet assembly of FIG. 9;

FIG. 11 is an isometric view of a tourniquet actuation means shown in FIG. 9, the tourniquet actuation means being shown in an open position ready to receive the tourniquet;

FIG. 12 is a section view of a torque limiting apparatus for use with the torque actuation means shown in FIG. 9;

FIG. 13 is a detail view of the torque limiting apparatus shown in FIG. 12;

FIG. 14 is a detail view of a tourniquet interlock and release;

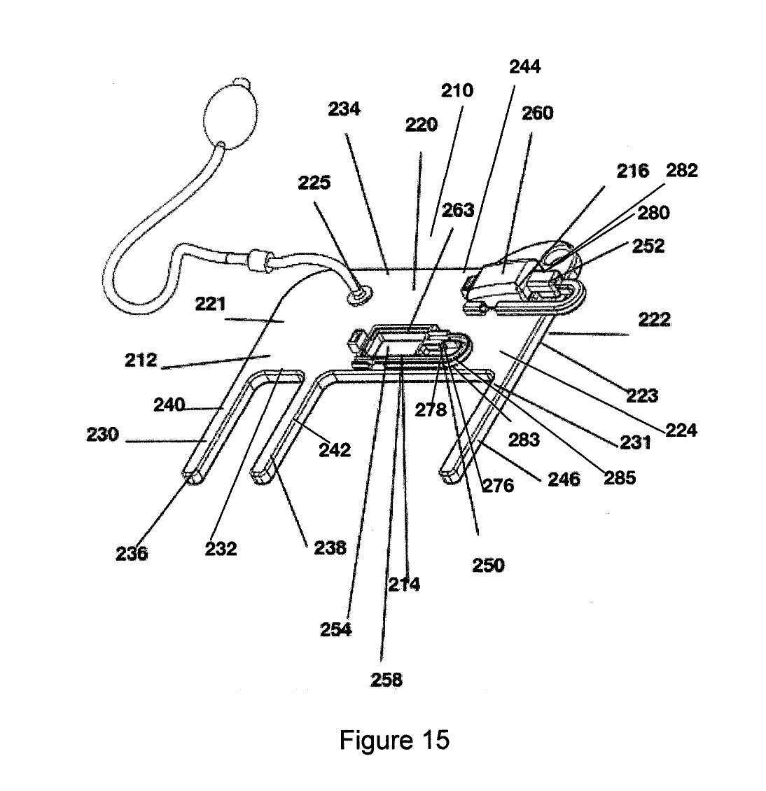

FIG. 15 is an isometric view of a cannula retainer in accordance with a preferred embodiment of the present invention from above and a front end;

FIG. 16 is a plan view of a penetration closure assembly being a component of a preferred embodiment of the present invention;

FIG. 17 is a side elevation section view along A-A of a locking window being part of the penetration closure assembly shown in FIG. 15;

FIG. 18 is a side elevation section view along BB of the window cap in the penetration closure assembly shown in FIG. 15;

FIG. 19 is a side elevation view of detail C from FIG. 16;

FIG. 20 is a side elevation view of detail D from FIG. 17; and

FIG. 21 is an exploded isometric view from above and one side of the penetration closure assembly shown in FIG. 15.

### DETAILED DESCRIPTION OF PREFERRED EMBODIMENTS

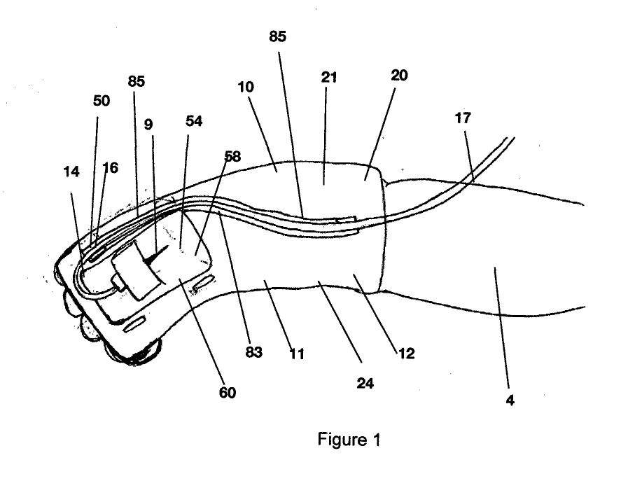

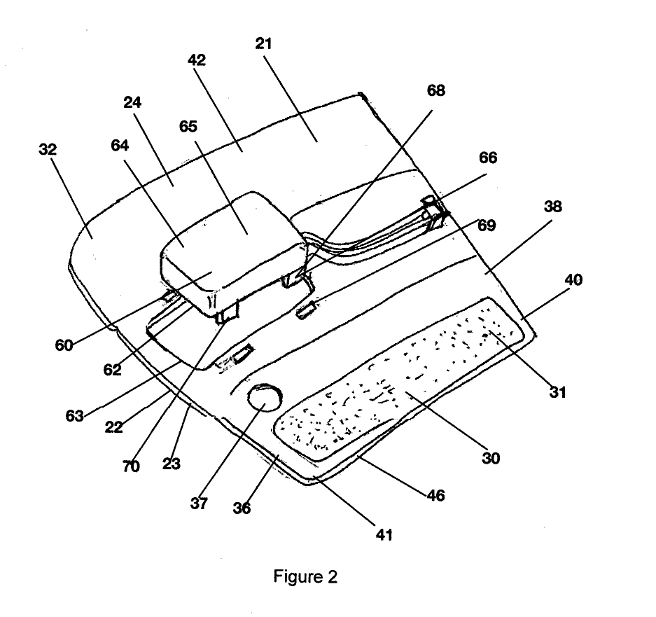

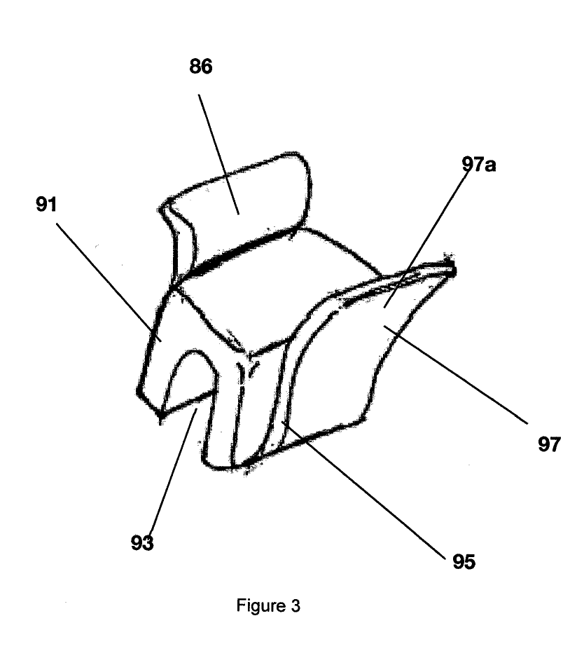

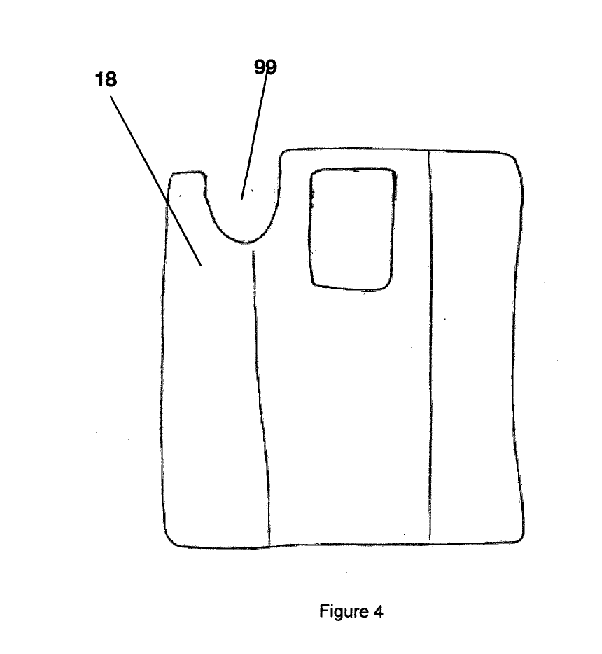

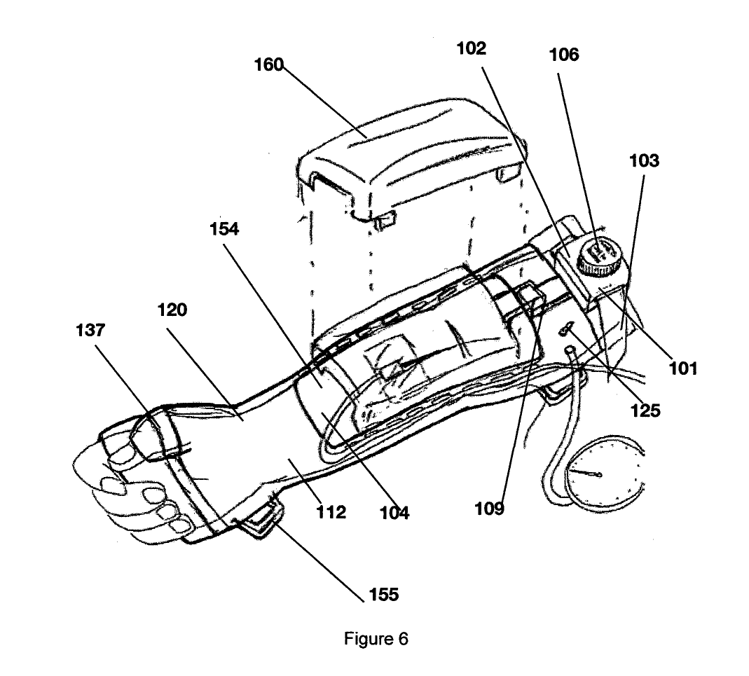

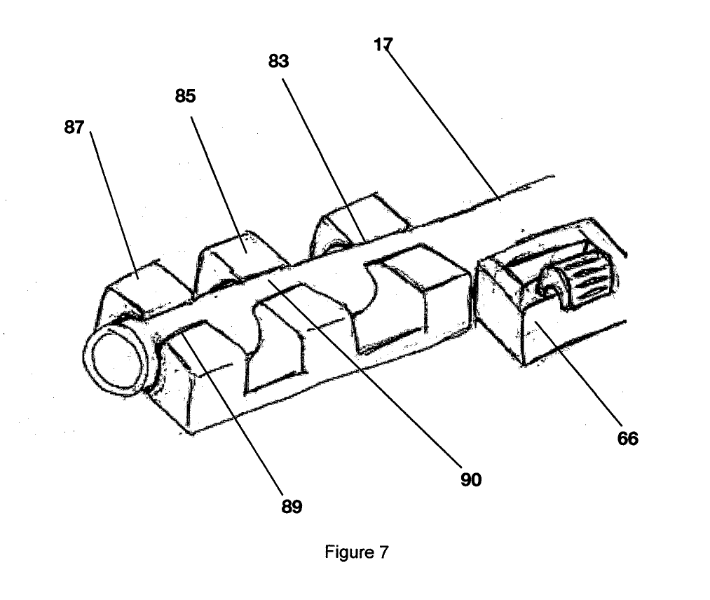

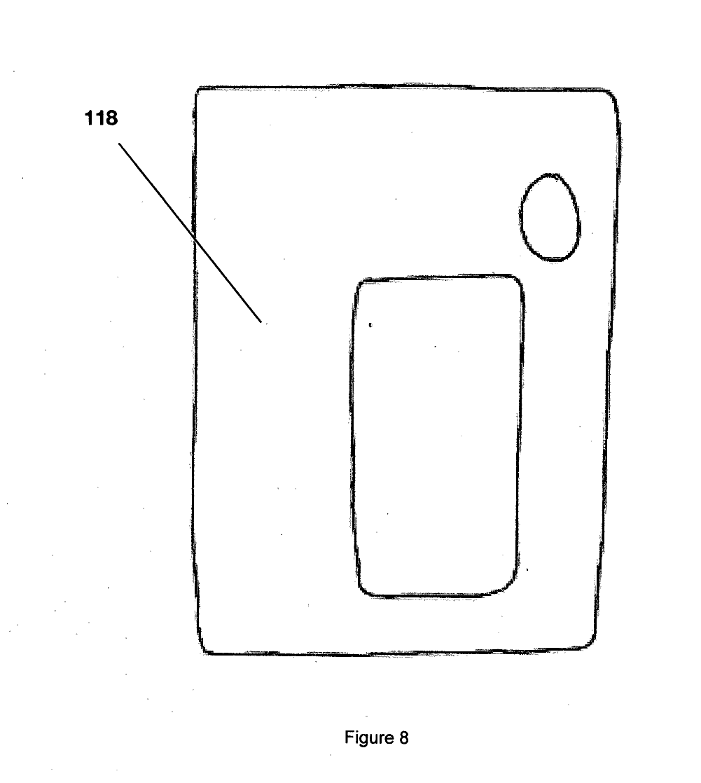

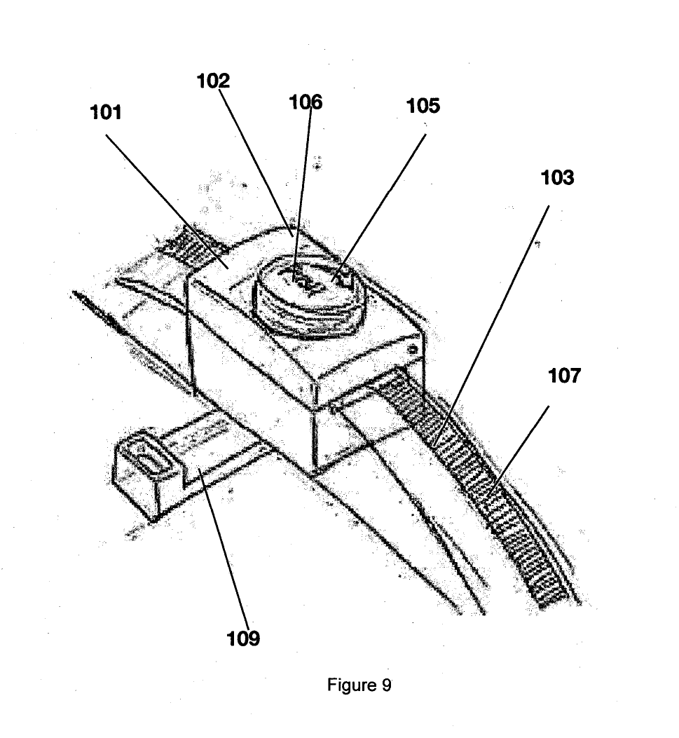

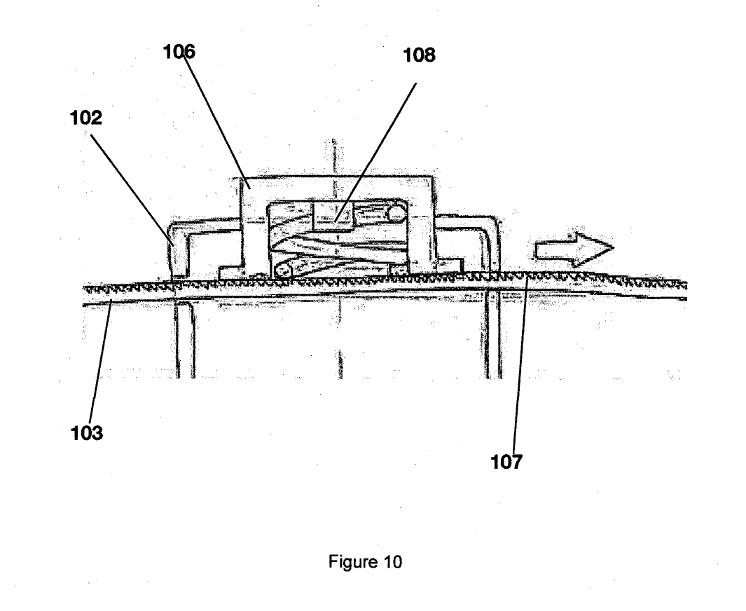

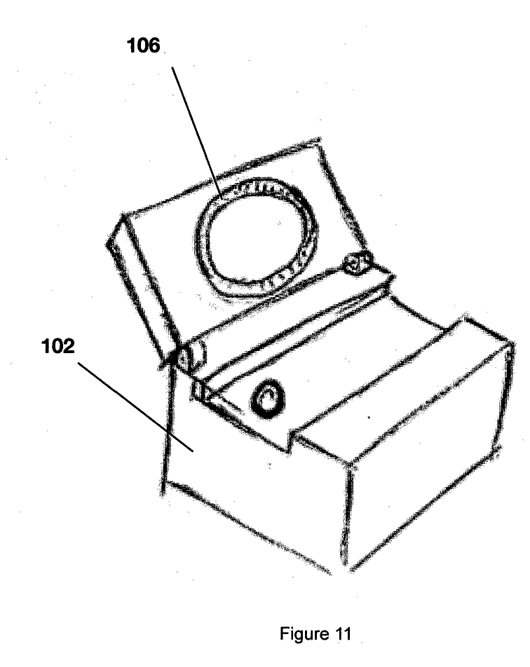

Referring to FIGS. 1 to 13 there is shown an embodiment of an intravenous cannula retainer generally indicated at 10. The intravenous cannula retainer 10 comprises a cuff 11 for supporting at least a portion of an arm of a patient. The cuff 11 is in the form of a splint 12 suitable for immobilising at least a portion of a limb of a patient (not shown), the splint 12 including a cannula retaining zone 14.

The arrangement of the intravenous cannula retainer 10 is such that the cannula retaining zone 14 in use inhibits access to a cannula 9 disposed within a blood vessel of a patient and thereby facilitating the retention of the cannula in the limb, the arrangement also locking a cannula line in a cannula line lock 16, one and both features inhibiting removal of the cannula 9 from the blood vessel of the patient. The splint 12 also, in use, being held against the arm and being provided with locks, also is inhibited from removal.

The cuff 11 includes a support curtain 20 for at least partially enveloping the limb of the patient in one or more selected positions.

The support curtain 20 may include battens (not shown) or other elongate members to facilitate support of the limb in one or more selected positions.

In the embodiment shown in FIGS. 1 to 13 the support curtain 20 facilitates the immobilisation of the limb in each of the one or more selected positions. One of the selected positions includes a position wherein the cannula can be inserted into a vein extending along the limb. Another of the selected positions includes a limb position in which fluid freely flows through the cannula and the limb is comfortable.

The support curtain 20 includes reinforcing elements (not shown). The reinforcing elements may, as described above, include battens or other elongate elements. The embodiment shown has reinforcing elements in the form of granules (not shown). The support curtain 20 includes at least two layers of curtain material 21, 22 and a splint void 23 therebetween. The curtain material is a tear-resistant polymer 24. The curtain material is strengthened by strengthening elements such as fibres. The granules are disposed in the void 23 between at least two of the at least two layers 21, 22 of curtain material.

The curtain material is airtight so as to be able to hold a vacuum in the splint void 23. The support curtain 20 includes a valve 25 in order to facilitate extraction of air from the splint void 23 in order to cause the granules to abut one another and to reduce movement between the granules and between the granules and the inside of the void once they are all in an abutting relationship. This arrangement in use facilitates immobilisation of the limb in the one or more selected positions by inhibiting flexibility of the support curtain 20.

The support curtain 20 includes a closure 30 to close the support curtain about the limb in the selected position. The closure 30 includes one or more fasteners 31 to fasten a first side 32 of the support curtain 20 to a second side 34 of the support curtain 20. The fasteners 31 include a lock 36 to inhibit their release by the patient whose limb is immobilised and wearing the cannula retainer 10, and/or to inhibit their release by anyone, for that matter.

The support curtain 20 includes a thumb spice 37 to support and/or immobilise the thumb in a support position.

The lock 36 includes elongate a cuff extension 38 extending from the first side 31 of the support curtain 20. The cuff extension 38 is rigid, either permanently, or may be actuated so as to move between a rigid form and a flexible form. The elongate straps 38 include hook and/or loop fasteners 40 on one side 41 thereof for fastening to hook and/or loop fasteners on the other side 32 thereof. The cuff extension 38 in the embodiment shown is integral with the support curtain 20, the arrangement being that the extension includes two layers of the support curtain material just the same as the cuff itself and is filled with granules in a strap void 46 therebetween, the extension void 46 being in fluid communication with the splint void 23. The advantage of the extension having an extension void 46 and hook and loop fasteners 40, 42 on one side is that the extension when the air is withdrawn from the voids 46, 23, the extension 38 is increased in rigidity and in use are thus inhibited from being peeled away from the support curtain 20, so as to form a kind of locking fastener 31, 36. When air is reintroduced back into the extension void 46, the extension 38 becomes flexible again and is less of a hazard to the patient and the carer.

There is provided a liner 18 to facilitate patient comfort when the limb is immobilised in the splint 10. The liner 18 includes a liner penetration or cutaway 99 for the thumb which aligns with the thumb spice when assembled to the curtain 20.

The cannula retaining zone 14 includes a cannula mount 60. The cannula mount 50 includes cannula penetration 54 for allowing a cannula to penetrate the support curtain 20. The cannula penetration 54 is an aperture 58. The aperture 58 is about 100 mm×100 mm in size and extends from one layer 21 of the support curtain 20 to the other 22.

The cannula mount 50 includes a cannula penetration closure 60. The cannula penetration closure 60 includes a closure rim 62 at least partially surrounding an aperture rim 63 of the aperture 58.

The penetration closure 60 includes a cover 64 for covering the cannula penetration 54. The cover 64 includes a window cap 65. The window cap 65 is transparent or translucent to allow inspection of the patient's limb disposed in use under the aperture 58. The cap is a cup shape to provide some volume under the window cap 65.

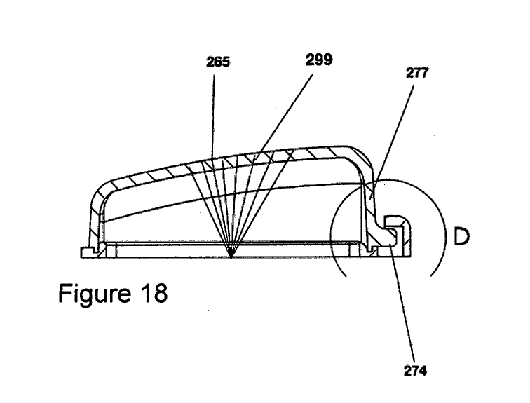

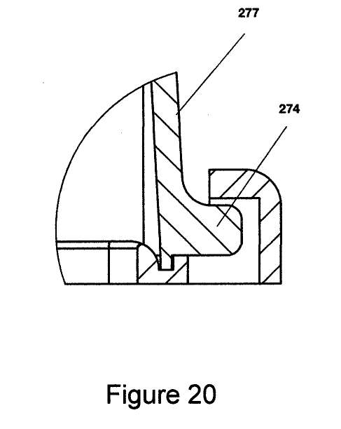

The window cap 65 includes a window cap retainer 66, shown in detail in FIGS. 17 and 19, with like numerals depicting like parts. The window cap retainer 266 includes a keeper 268. The keeper 268 is adapted to be received in a catch 269 disposed on the closure rim 262. The keeper 268 is removably connectable to the catch 269 and the arrangement is such that the keeper 268 can be released by operating a child-resistant lock 270. The child-resistant lock 270 includes a flexible keeper arm 271 and a rib 272 extending therefrom so as to be caught by the catch 269 in a locking position. The catch 269 includes a cross-member 273 so that the rib 272 can be released from the catch 269 only by a finger press under the cross-member 273, the cross-member 273 being hidden from view from the patient, who is likely to be a child.

The window cap retainer 266 includes locating lugs 274 to facilitate locating the window cap 265 on the closure rim 262.

Returning to FIGS. 1 to 12, the penetration closure 60 includes a cannula tube receiver 83. The cannula tube receiver 83 includes a tube mount 85. The tube mount 85 extends along one portion of the rim. The tube mount 85 includes a tube channel 87 for mounting a tube 17 therealong. The tube channel 87 includes one or more retaining ribs 89 along an open edge 90 so as to retain the tube within the tube channel 87.

A line lock 86 is provided to lock the tube 17 within the tube channel 87. The line lock 86 includes a main lock body 91 including a tube receiver 93 and a locking means 95. The locking means 95 includes at least one resilient locking member 97. The resilient locking member includes an actuator and a detent. The resilient locking member includes a sigmoid element 97a so that the base of the sigmoid can be received by the tube channel 87 or a detent receiver and the top of the sigmoid 97a can be the actuator to retract the detent or the base of the sigmoid from the tube channel. The action to remove the line lock 86 is to squeeze the two upper portions of the sigmoid together and lift the lock body 91 out of the tube channel 87.

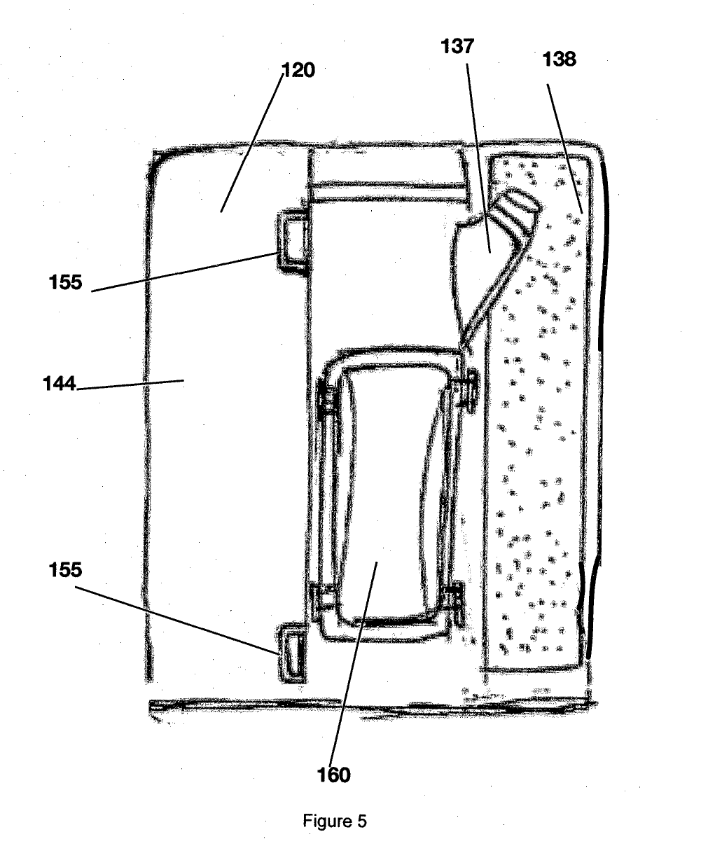

In the embodiment shown in FIGS. 5 to 13, all features like those in the embodiment described above (and below, for that matter) are denoted with like numerals.

In FIGS. 5 to 13, a cannulation assembly is provided, which includes a tourniquet assembly 101. The tourniquet assembly 101 includes a belt 103 for encircling the arm 104 of the patient and restricting blood flow through the arm 104 past the tourniquet, so that in use a blood vessel becomes more prominent.

A tourniquet actuation means 102 is also provided. The tourniquet actuation means 102 includes a belt advancing means 105 to tighten the belt 103 around the arm 104. The belt advancing means 105 includes a dial 106 twists to advance the belt 103 through the belt advancing means. The belt includes a rack 107 which engages with cooperating teeth on the dial 106.

The tourniquet assembly includes a tourniquet release 108. The tourniquet release is disposed on the dial. The tourniquet release 108 is actuated by pressing the dial 106, in a similar way to certain kinds of ball-point pen retraction means.

The tourniquet release is in the embodiment shown also in an interlock form, in which a tourniquet release arm 109 is actuated by a corresponding tourniquet release arm 109a disposed on the penetration closure 160.

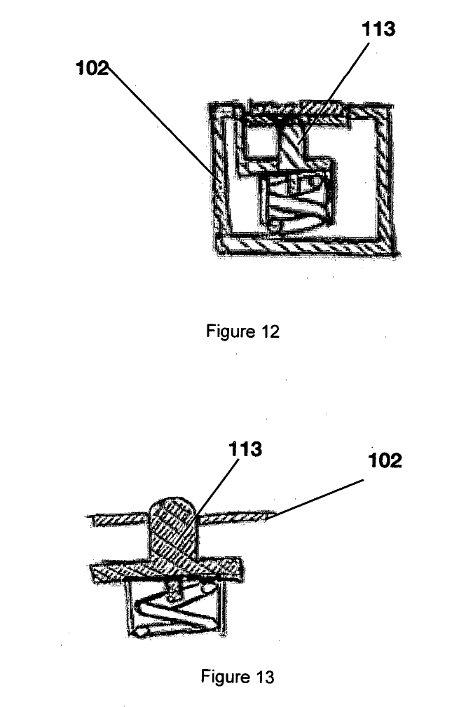

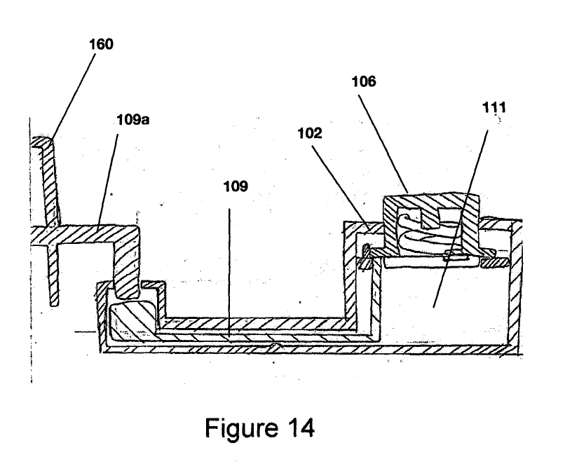

A torque limiting apparatus 111 is also provided and shown in FIGS. 12 and 13. A release element 113 is disposed in the tourniquet actuation means 102, which is disposed on the support curtain 120, the release element 113 biased into a keeping position. The release element 113, if the support curtain 120 is pressed against the base of the tourniquet actuator such that it may in use be pushed upwards against the biasing spring, may press upwards against the dial and release the dial and cooperating pinion from its hold on the belt with rack.

In operation a carer holds a limb of a patient in a selected position suitable for cannulation. The carer applies the splint to the arm and locates it in a suitable position by inserting the thumb of the patient in the thumb aperture 99.

The support curtain 120 is wrapped around the limb and the locking fasteners 31 are fastened to the other side 144. Air is evacuated from the void 123 by valve 125 and the granules abut one another and the walls of the void 123. Air may be evacuated from the void 123 by hand, by an electric pump, or via a one-shot gas canister which when opened blasts air along a venturi which draws air from the inside of the void 123. In any event, the splint 112 becomes somewhat rigid and inhibits movement of the limb, and inhibits its own removal from the limb because the layer 22 grips the skin of the limb, and the support curtain extension 38 cannot be peeled away because they are rigid. That is, the full hook and loop force must be applied all at once, rather than a small bit at a time, as in peeling. This inhibits removal of the support curtain extension lock 38.

If the patient becomes uncooperative or has the potential to be so, restraints 155 can be utilised with straps (not shown) to further immobilise the arm of the patient.

If the embodiment shown in FIG. 5 is being used, the carer then inserts the belt into the tourniquet actuator and then twists the dial to tighten. If the operator tightens the dial too far, the torque limiter releases the dial from the belt and the carer must start the tightening process again with the dial. The vein or artery becomes more prominent; a trocar needle and cannula is inserted into the vein of the patient.

If the embodiment in FIGS. 1 to 4 is being used, then the cannula will already be in place and the cuff 20 will be applied over the cannula, locking the tube 17 into the cuff 20.

The tube 17 is placed in the tube channel 87. The tube lock is placed in the tube channel 87. The window cap 65 is placed, lugs first, into the rim and is then locked in place. The interlock arm on the window cap 65 releases the tourniquet.

To remove the splint 12, air is introduced back into the void 23 by opening the valve 25.

### Alternative Embodiment

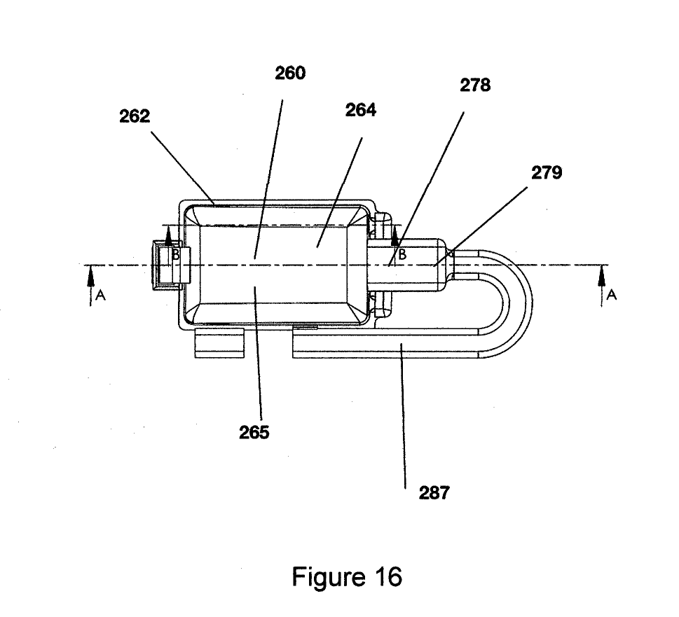

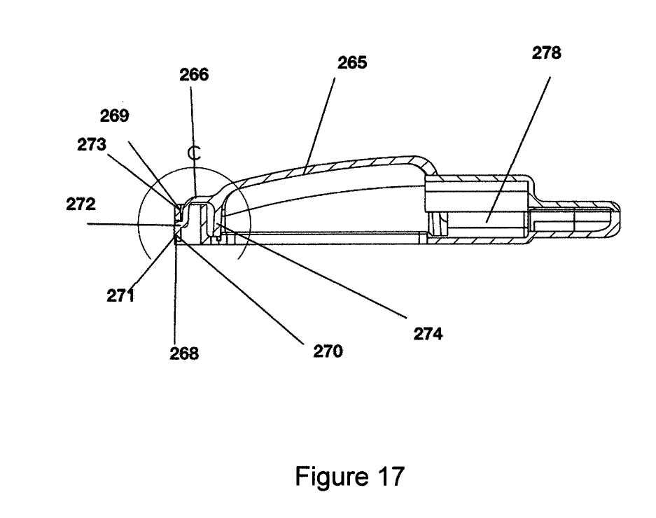

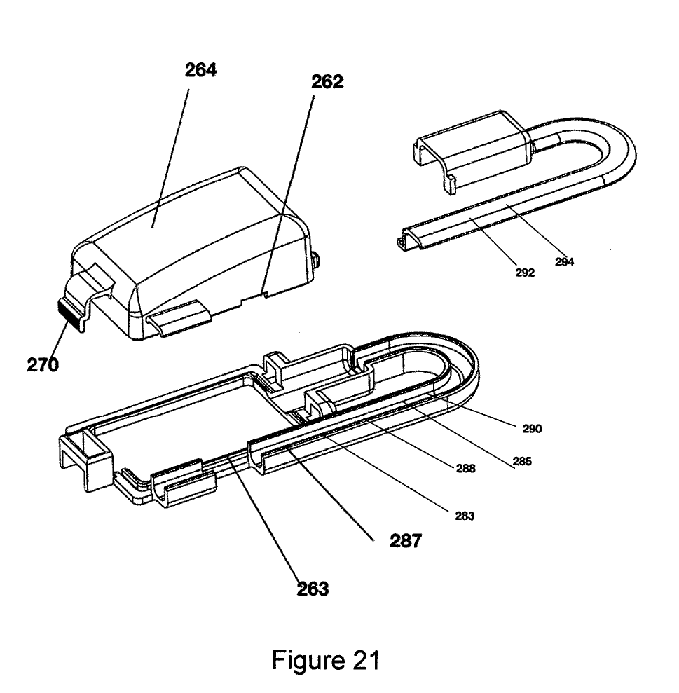

Referring to FIGS. 15 to 20 there is shown an alternative embodiment of a cannula retainer generally indicated at 210. There are many similar features to those described in other embodiments and like numerals described in those embodiments denote like features. The cannula retainer 210 comprises a splint 212 suitable for immobilising a limb of a patient (not shown), the splint 212 including two cannula retaining zones 214 and 216.

The arrangement of the cannula retainer is such that the cannula retaining zones 214 and 216 in use inhibit access to a cannula (not shown) disposed within a blood vessel of a patient and thereby facilitating the retention of the cannula in the limb and inhibiting removal of the cannula from the blood vessel of the patient. The splint 212 also, in use, being held against the arm and being provided with locks, also is inhibited from removal.

The splint 212 includes a support curtain 220 suitable for supporting the limb of the patient in one or more selected positions. The support curtain 220 is for at least partially enveloping the limb of the patient in each of the one or more selected positions.

The support curtain 220 may include battens (not shown) or other elongate members to facilitate support of the limb in one or more selected positions.

In the embodiment shown in FIGS. 15-20 the support curtain 220 facilitates the immobilisation of the limb in each of the one or more selected positions. One of the selected positions includes a position wherein the cannula can be inserted into a vein extending along the limb. Another of the selected positions includes a limb position in which fluid freely flows through the cannula and the limb is comfortable.

The support curtain 220 includes reinforcing elements (not shown). The reinforcing elements may, as described above, include battens or other elongate elements. The embodiment shown has reinforcing elements in the form of granules. The support curtain 220 includes at least two layers of curtain material 221, 222 and a splint void 223 therebetween. The curtain material is a tear resistant polymer 224. The curtain material is strengthened by strengthening elements such as fibres. The granules are disposed in the void 223 between at least two of the at least two layers 221, 222 of curtain material.

The curtain material is airtight so as to be able to hold a vacuum in the splint void 223. The support curtain 220 includes a valve 225 in order to facilitate extraction of air from the splint void 223 in order to cause the granules to abut one another and to reduce movement between the granules and between the granules and the inside of the void once they are all in an abutting relationship. This arrangement in use facilitates immobilisation of the limb in the one or more selected positions by inhibiting flexibility of the support curtain 220.

The support curtain includes a closure 230 to close the support curtain about the limb in the selected position. The closure 230 includes one or more fasteners 231 to fasten a first side 232 of the support curtain 220 to a second side 234 of the support curtain 220. The fasteners 231 include a lock 236 to inhibit their release by the patient whose limb is immobilised and wearing the cannula retainer 210, and/or to inhibit their release by anyone, for that matter.

The lock 236 includes elongate straps 238 extending from the first side 232 of the support curtain 220. The elongate straps 238 may be actuated so as to move between a rigid form and a flexible form. The elongate straps 238 include hook and/or loop fasteners 240 on one side 242 thereof for fastening to hook and/or loop fasteners on the other side 244 thereof. The elongate straps 238 in the embodiment shown are integral with the support curtain 220, the arrangement being that the straps include two layers of the support curtain material and are filled with granules in a strap void 246 therebetween, the strap void 246 being in fluid communication with the splint void 223. The advantage of the strap having a strap void 246 and hook and loop fasteners 240, 242 on one side is that the strap when the air is withdrawn from the voids 246, 223, the straps 238 are increased in rigidity and in use are thus inhibited from being peeled away from the support curtain 220, so as to form a kind of locking fastener 231, 236. When air is reintroduced back into the strap void 246, the strap 238 becomes flexible again and is less of a hazard to the patient and the carer.

There is provided a liner (similar to 118) to facilitate patient comfort when the limb is immobilised in the splint 210. The liner includes a liner penetration for the thumb. The support curtain 220 includes a thumb aperture 237 for receiving a thumb. The thumb aperture may be a tube for inhibiting supination, pronation and flexion.

The cannula retaining zones 214, 216 include a cannula mount 250, 252. The cannula mount 250, 252 include respective cannula penetrations 254 for allowing a cannula to penetrate the support curtain 220. The cannula penetration 254 is an aperture 258. The aperture 258 is about 100 mm×100 mm in size and extends from one layer 221 of the support curtain 220 to the other 222.

The cannula mount 250 includes a cannula penetration closure 260. The cannula penetration closure 260 includes a closure rim 262 at least partially surrounding the rim 263 of the aperture 258.

The penetration closure 260 includes a cover 264 for covering the cannula penetration 254. The cover 264 includes a window cap 265. The window cap 265 is transparent or translucent to allow inspection of the patient's limb disposed in use under the aperture 258. The cap is a cup shape to provide some volume under the window cap 265.

An interior face of the curtain of the splint 210 is in the form of temperature or pressure-responsive material so that it responds to an increase in pressure by changing colour. Advantageously, the interior and/or exterior layer of the splint 210 then can be seen changing colour through the penetration closure 260 and the window cap 265. Thus the material itself can act as an infection alert and/or a compartment syndrome alert, rather than relying on the patient themselves, who may not or may not be able to raise an alarm in relation to those serious conditions, or who may raise the alarm unnecessarily.

An outer layer of the curtain may be tear- resistant or cut-resistant so that a patient or an accomplice may be inhibited from cutting through the splint 210 with a knife or other instrument, or even fingers.

The window cap 265 includes a window cap retainer 266. The window cap retainer 266 includes a keeper 268. The keeper 268 is adapted to be received in a catch 269 disposed on the closure rim 262. The keeper 268 is removably connectable to the catch 269 and the arrangement is such that the keeper 268 can be released by operating a child-resistant lock 270. The child-resistant lock 270 includes a flexible keeper arm 271 and a rib 272 extending therefrom so as to be caught by the catch 269 in a locking position. The catch 269 includes a cross-member 273 so that the rib 272 can be released from the catch 269 only by a finger press under the cross-member 273, the cross-member 273 being hidden from view from the patient, who is likely to be a child.

The window cap retainer 266 includes locating lugs 274 to facilitate locating the window cap 265 on the closure rim 262.

The cannula penetration closure 260 includes a cannula mounting base 276. The cannula mounting base 276 includes a cannula rest 278 for resting a portion of the cannula thereon. The cannula rest 278 is disposed flat and against the skin of the patient so that the cannula may remain flat and horizontal so as to inhibit breaking through a wall of a blood vessel when the cannula is on the cannula rest 278. The cannula rest 278 extends from a portion of the rim and in one embodiment is integral therewith, so that in use a rear wall 277 of the window cap 265 when in the closed position can inhibit release of the cannula from the cannula rest 278. The cannula rest 278 includes a cannula rest cradle 279 to facilitate immobilisation and support of the cannula when in use.

The cannula rest 278 includes locating lug receivers 281 to receive the locating lugs 274 and facilitate locking and retention of the window cap 265.

The penetration closure 260 includes a cannula rest cover 280 for covering the cannula rest 278. The cannula rest cover 280 includes a cannula rest cap 282 and is preferably transparent or translucent to facilitate monitoring of the retention of the cannula.

The cannula mounting base 276 includes a cannula tube receiver 283. The cannula tube receiver 283 includes a tube mount 285. The tube mount 285 extends along one portion of the rim, either on the outside as shown in the drawings or on the inside of the window cap 265 for additional security and to further inhibit removal of the tube. The tube mount 285 includes a tube channel 287 for mounting a tube therealong. The tube channel 287 includes a break 288 so as to facilitate locking of the tube (not shown) within the tube mount 285. The tube channel 287 includes one or more retaining ribs 289 along an open edge 290, either on the inside edge or the outside edge of the window cap 265 so as to retain the tube within the tube channel 287.

The cannula penetration closure 260 includes a tube channel cover 292. The tube channel cover 292 includes an elongate cover wall 294 extending along the tube channel from a rear portion of the cannula rest cover to an end of the rim.

In operation a carer holds a limb of a patient in a selected position suitable for cannulation. The carer applies the splint to the arm and locates it in a suitable position by inserting the thumb of the patient in the thumb aperture.

The support curtain 220 is wrapped around the limb and the locking fasteners 231 are fastened to the other side 244. Air is evacuated from the void 223 by valve 225 and the granules abut one another and the walls of the void 223. Air may be evacuated from the void 223 by hand, by an electric pump, or via a one-shot gas canister which when opened blasts air along a venturi which draws air from the inside of the void 223. In any event, the splint 212 becomes somewhat rigid and inhibits movement of the limb, and inhibits its own removal from the limb because the layer 222 grips the skin of the limb, and the straps 238 cannot be peeled away because they are rigid. That is, the full hook and loop force must be applied all at once, rather than a small bit at a time, as in peeling. This inhibits removal of the straps 238.

The cannula is inserted into the penetration 254. The cannula is inserted into the vein of the patient. The tube is placed in the tube channel 287. The cannula is mounted onto the cannula rest 278 and then the cannula rest cover is placed on the cannula rest 278. The tube channel cover 292 is integral with the cannula rest cover and is placed on the tube channel 287. The window cap 265 is placed, lugs first, into the cannula rest and is then locked in place with the keeper arm 271.

To remove the splint 212, air is introduced back into the void 223 by opening the valve 225.

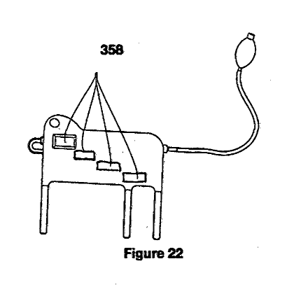

In other embodiments shown, such as those shown in FIG. 21, there are a plurality of apertures 358 disposed in a cascading arrangement across the curtain. In the embodiment shown there are four apertures 358 shown, indexed across the curtain and along the curtain so as to support a plurality of positions for the cannula to be inserted.

One of the arrangements includes a pair of apertures 358, one in opposite quadrants. In that arrangement, the wrist of the patient may be disposed at either end and the curtain may be rotated and flipped so that the window may support a cannula penetration at the elbow or the wrist, on the inside or the outside of the arm or palm.

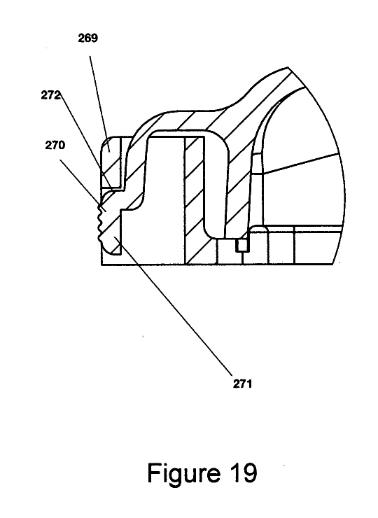

In one embodiment shown in FIG. 18, there is provided a mounting frame on the apertures 358. The mounting frame is for reinforcing the edge of the aperture 358 and for facilitating secure mounting of the window cap. The mounting frame is hinged so that the mounting frame can facilitate setting of an arm of a patient in a selected angular position when in the splint 310. In this embodiment the window cap 365 is also hinged by having corrugations 299 at an intermediate position so that the window cap can also hinge to accommodate the angled arm position.

It is to be understood that although there are several distinct aspects of the invention defined individually herein, they may be combined with one another so that optional aspects of the invention may be provided in combination with any one aspect hereindefined and described.

The word ‘comprising’ and forms of the word ‘comprising’ as used in this description do not limit the invention claimed to exclude any variants or additions.

Modifications and improvements to the invention will be readily apparent to those skilled in the art. Such modifications and improvements are intended to be within the scope of this invention.

1. An intravenous cannula retainer for retaining an intravenous cannula on a patient, the intravenous cannula retainer comprising:
a cuff including a support curtain suitable for at least partially supporting at least a portion of a limb of a patient, the cuff including at least one intravenous cannula retaining zone comprising a cannula penetration for providing access through the cuff to the limb of the patient, and a frame connected to the cuff and extending around the cannula penetration zone for reinforcing an edge thereof;
a cannula penetration closure being a cover for inhibiting access to the intravenous cannula, the cover configured to be removably mounted on the frame,
wherein the frame and the cover are configured to flex about one or more axes to accommodate contours of the patient over convex and concave cannulation sites.

2. The intravenous cannula retainer in accordance with claim 1 wherein the cuff is in the form of a splint for immobilising at least a portion of the limb.

3-4. (canceled)

5. The intravenous cannula retainer in accordance with claim 1 wherein the support curtain includes battens or other elongate members to facilitate support of the limb in the one or more selected limb positions.

6-15. (canceled)

16. The intravenous cannula retainer in accordance with claim 1 wherein the support curtain includes at least one closure to close the support curtain about the limb in the selected position.

17. The intravenous cannula retainer in accordance with claim 16 wherein the closure includes one or more fasteners in the form of elongate hook and loop straps to fasten one side of the support curtain to another side of the support curtain.

18-35. (canceled)

36. The intravenous cannula retainer in accordance with claim 1 wherein the cover includes a window cap to facilitate a selected amount of space under the window cap.

37. The intravenous cannula retainer in accordance with claim 36 wherein the window cap is transparent or translucent to allow inspection of the portion of the patient's limb disposed in use under the aperture.

38. (canceled)

39. The intravenous cannula retainer in accordance with claim 36 wherein the window cap includes a window cap retainer for retaining the window cap on the frame.

40-44. (canceled)

45. The intravenous cannula retainer in accordance with claim 39 wherein the window cap retainer includes locating lugs to facilitate locating the window cap on a selected portion of the frame.

46-54. (canceled)

55. The intravenous cannula retainer in accordance with claim 1 wherein the frame includes a cannula tube receiver which includes a tube mount extending along one portion of the rim for mounting a cannula tube.

56-58. (canceled)

59. The intravenous cannula retainer in accordance with claim 55 wherein the tube mount includes one or more retaining lugs so as to retain the tube within the tube mount.

60. The intravenous cannula retainer in accordance with claim 59 wherein the retaining lugs are arranged in a sinusoidal or sawtooth channel or in the form of discrete tube supports disposed in a sinusoidal arrangement on the support curtain so that the tube is directed to a sinusoidal mounting position.

61-66. (canceled)

67. The intravenous cannula retainer in accordance with claim 1 wherein the support curtain is long enough to extend when disposed on a limb, along the limb far enough to provide immobilisation of a wrist.

68. The intravenous cannula retainer in accordance with claim 67 wherein the cannula penetration is disposed on a rear portion of the support curtain so that the cannula penetration is disposed adjacent the back of a patient's hand when in an immobilising position on the patient's limb.

69. The intravenous cannula retainer in accordance with claim 67 wherein the support curtain extends along the limb far enough to provide immobilisation of an elbow.

70. The intravenous cannula retainer in accordance with claim 67 wherein the support curtain extends far enough along the limb to provide immobilisation of an elbow, arm and wrist joints.

71-88. (canceled)

89. The intravenous cannula retainer in accordance with claim 1 wherein the cover includes a cover hinge to facilitate hinging about the one or more axes.

90. The intravenous cannula retainer in accordance with claim 90 wherein the hinge includes a plurality of corrugations to facilitate rotation about the one or more axes.

91. The intravenous cannula retainer in accordance with claim 1 wherein the frame includes a frame hinge at an intermediate location along one or more frame elements.
